SEQUENCE LISTING (29 sequences)

SEQ ID NO: 1 - protein, 258 aa, Propionibacterium acidipropionici
MSDGGISPGDRLARAHSFGEAAADYQRYRPDYPIEAVTYLVAGTPAGGRVLDLGAGTGKLTDRLVALGFEVVAVDPSAQMLAELSRRRPDVDCRVGTGESLPLPDSCVDGVVCGQAWHWMDAGAVGRELARVMRPNGSLGLAWNTDHTDTGWLARIEAIRNVPRGAELNRGPDRTPVSPGQGWSPFTRHDVDWTRTMTKEDFLALWRTHSQWLTATEEQRIRWMSGWRDVLATDPQVATLDTVSIPMTTECWVTRPGG

SEQ ID NO: 2 - protein, 412 aa, Propionibacterium acidipropionici
MLGDATGRLRVGEQKQAGRGLSRGLLLLMATATGLAVGGNYLNQPLIDEIARHFSVSVSTAATSVTVTQFAYALGLVLFVPLGDMINRRKLAVTLFLVSAAGLLTAAVSGSFAVMMIGTAIASLFSVAAQVLVPFASELAAPGRGGAAVGTMMTGLLTGILVARAVSGMLSLVGGWKTAYWVLGVLLLVMAATLWKMLPDVPVAESFSLTRVPASMGRAWMRYPKVRSRAVISALLFASVSACFATMTPLLAGPPHRLGPGVIGILGLLGLVGAFAAGPVGRMADRGLGNRTVVLGLVILAAGWASMWFATGSVVMFGIGFILTDLGLQSAHVTNMNVVYAQEPALRSRLNSLYMTMYFIGGSVGSAVAVGLWSRFAWHGVVIAALAFVTAAGVVFALERLSDRRPRIASQG

SEQ ID NO: 3 - protein, 264 aa, Propionibacterium acidipropionici
MALLIILIILVVVIGGGVGLFIAPYNSFVKLRNTIQESWRQVDVELNRRYELIPNLVETVRAGAAHERNTLEEVTRLRNQAVAMATNSQGNTPDPQRSQIESQLSGAVHNLVAQVEAYPELRSNTNFLELQRELSDTEDRIAAGRRYYNANVKTYNTKVESFPSNLVAGMFHFEKASYFQVDDPAVRSAPGVNFGEISQRPEAQQNQGQAPQIGQGNPAQAPGYAAPQASGQLPDPQAARQPDPSQQPWGTQGKPGDQGNQNSQ

SEQ ID NO: 4 - protein, 166 aa, Propionibacterium acidipropionici
MSANPVPQVPGTGTLLADVFRWHPAGVAVLTADGPSRPVGITVSSLASVSVAPPMVSVSMANSSTTLAALHLGGRAVVHLLDAGQEDLADSFARPGVPAVGIDWERTAENAPQLDVETPRLHAVVARMIDTGSATLVALTIERIEVGRRGSAALVRMGRGWYTLPR

SEQ ID NO: 5 - protein, 210 aa, Propionibacterium acidipropionici
MSEQTSPASPTPGPRPVTEIGTLPPIDVVEKRLPVDPATGRPRREILATIATICYILAAGASAVALARAWWGTINMRTFHLATNLMTWTDPRPGSLASVLLAALMMVIGGVMVAMPALLAVNTWLGRRWVRWGAIGGVAAAVLAVTLNPLAWISAPFSIAGGVMVWLPSTRRWFELWRQVRSEPEVERFTPRPITYGPVAKHMWPPGRRT

SEQ ID NO: 6 - protein, 390 aa, Propionibacterium acidipropionici
MSQGEPGYRRASLALLAAGLASFNALYCTQALMPTLTSQLGATPAQASLTVSAATGILAITILPVSVLSERFGRGRLMTISAMAAVVVGLLLPLAPSLGWLVVGRGLQGLLVAGVPATAMAWLSQEIHPRHLPRAMGLYVAGNTVGGLLGRLIPSGVLQFTGWRPALGIDMAFALVCTVAMVTLMPAERRFVPKQLRPGNELRTMGRQWADRRLAGLFGIGFIFMGVFVSLYDFLGYRLTARFGMPPSLIGLVFLLYLFGTLASARAGHLTATRGRGPAMLIGAAMAIVGMPLVASGLLWLTLPGVALFTYGFFTVHSVASGWVGALAPRARGEASGTYLACYYLGSSILGYLSGHVMHAFGWTGLVMWLVGLILIGCALSAMVVRSARS

SEQ ID NO: 7 - protein, 391 aa, Propionibacterium acidipropionici
MAATATRPPRDRRPSVVHLSTVHNRHDNRVFNKEARALVNAGYDFHLVISADADGVDDGIPVVGLHRTVGPRLRRIVAGQLEAWRVLGSLRPELLQIHDPELIPMALLWGRTHPCKVVYDAHEDLVGQIDTKPYLNRLTRPVARAAARCLVGMADRGADGIVAATEPVADRFRNPRIAVVHNYPWLANFTVDPAPVPGRLVYAGDLSQERKLSFMIDVVRALRATVPAAHLVLAGRALKGCGPVVEAGVAEGLVDYRGLVGPTEVPGVLASAQVGLVFLEPLPNYVRSLPTKLFEYMAAGVPFCASDFPAWSQMFSGYGAGAFADSESVETTANVLAGLLTDPQGCEQMGEAGRRAIGEGLTFEAQSRALLTLTEELLGCGAGPLGGGERQ

SEQ ID NO: 8 - protein, 451 aa, Propionibacterium acidipropionici
MGACAEALLWLLFIVLTFISTDQAIASDRAYPMLLVVLGVAGAGCLVLGVLRRREGRSAVSGSGIGRPGWRALGLTALPFLAMLCWAGITIPFATHVALSNRFGPLVHIRLPMASMVVPLVEAALVVLVAVGLVVGIGRRRLPEALWRAFLVLAASTLISIVWQVATRHAMVRRAVDGKLMWRTSTQLGGQATYHLALLLGIGVAVDAIRRRYRVGVSWLIIAGLGLAIVLSGSRAGLICLGLFCVALFIWGRPAGGRGGGGRRRTLVGVLGLAALAVVAGALLWLRGGALVDHDRAQTWKVAWRAVTADPTTVIVGRGYATIWPWFATETGIVPGAIHGLRPGPFGKSLVHAHNTVVQVGGELGIIGLVLLLVSVVGVAVLAFRGIHGRHLGICLALLASLPALVLDTYLVKNFQVSLVWWLVAAAVAVLMARPADHDQPTNSDQPADRP

SEQ ID NO: 9 - protein, 455 aa, Propionibacterium acidipropionici
MSRRAFRYRDTTPGTVLGVVAAMLVLRLPLSFLLFVVAPWFLILTGAIAKGGSVRCLPLKAMLGWCLVAATISIAVLHPGVAASTGNNVVIMIAVIGCTLVVHRGQAPGLTARRTLAGLYWGAGGVWLIAMGEMITGIKLLPILYPDANTVSYVQSSRFIVSATYPNINDFSVVLVMLVTAVVARMWFDRARGWRNAGRWLVLLTSLFMVVMSTSRGALVGCLAGVALLIVLNVRRLHPHALGVRAGLFGGGLIVFVGAVFFTSSYVQDHSTATRGQIFNNAMSMLAGSPADALLGYGSLASYQSAAKAAFGDVLMDPHNMLLEITLNYGVIALVLFIVVWLWVLMRGFLPRRPMADWQTAFGLTTVVLLPLLGVVPSSTLRYHVTWIYLAATTLLVAEGAEARTPTRPELSDEQPSGRELSGSDTSGATAADGDLGHAHDDHARDQAEHHSDHR

SEQ ID NO: 10 - protein, 437 aa, Propionibacterium acidipropionici
MARRHGLIGGWREPMGRQRVWLPVAGLSALLVVETFFDLRRSMTPSPWLWNSYLIVHLLAALACLVLILAGRDGNRFSRAGWIVIGFAAALMACSLVSAAVTPLPRVSYVTVPRAYLVVPTLTAAATLLLGAAVVRVLPDQPVHRVLWWPAALTLACAFAQWPRSAVVHGSPRLATGMGGSAVVHVPLLLATGVALAAFLAGWRRWWSLGLTVIGVAAVVLTGSRSGVVCLVLAGVVVGLQWLRSRRAWLVAGAAVVALGIVVAAVPMLHRLLNPTDELRAKNLETALGVWTETPKHLLLGVGSGRLWPWYAFDSHLLRTPWRGMVTTEWGPALNSAHSTFLQVLVELGLLGMLLLIPVVVVPVVVLARRLWPGLRGAARPVPDTVPLIALVATVPAFFLDTYLLKNYGASLWWWLVVLRCLRRAQSAPSTSVQNRG

SEQ ID NO: 11 - protein, 767 aa, Propionibacterium acidipropionici
MAEPRHAGRRGARHGKAKRRKGSPFGRFVKRTLFSLLILVLVLAIAAGVGAIVFYNRTNLPDPNKDFQTNTSFIYFNDSKTKLGSLSVQNRQTISYEQMPKSIKDAAISAENRTFWEDQGISIGGIVRAAWTIARGGEMQGGSTITQQYIKILYLSQDRTMQRKLKELVLAVKMGKQVPKEDILAGYLNTIYFGRGAYGIQAAAKSYFNVDASKLTVPQSAVLASILNNPTLFDASGGTKARERLLNRYRYVLDGMLEAGNITQAQHDEYSRKLPAFPEVPINNRWGGTNGYLLKMVQNELLDDGFTDSQINGGGLKVTTTFDPAAQKAAVATGQKYKKLAGSNAGKNGAKNLHPAIASVKVGTGEVLALYGGDDYITSTRSWALQARPAASTFKTYAVIAGMRNGFSLKSKLNGDTFTPQGDSVPIRNEFSEQYGDVTLQKATEDSINTAFVDMMTQIDNGPQAMLKAANDAGVPKGSGWDLNNRMPLGVAEVSPLDQATGYATIANEGKYVPSHVVAKVTDSSGKTLYTAKTTGKQTIQKDIAHDTTYALENVVNEGTGSAVSNLGYPVAGKTGTNGVKDDITSAWFVAYTRQISTAVMYVAGDSGNADLDPYAAPGDATFFGGTYPARTWADYMKVAMKGLPAKDFPDPDWVNLSGNHYGDTQRETLRTPTPTPTPTQAPTTATSQPTVTATATQPTQQPTTQPPATVQPTQEPTTSTATNRPTSTATNGSGGDGDGGGGDGGDGGGGNGNNGNGANAAPGGNG

SEQ ID NO: 12 - protein, 297 aa, Propionibacterium acidipropionici
MVSSTPPAEVDITVDLVHALLAEQHPDLADRRLKVVANGWDNVIVRIGEDLVARLPRRQLAADLILHEQRWLPELARGLPILVPAPVRQGRPGHGYPWFWSICPWFEGEVAADVPLADPVREADRLGAFVHAFHRPAPPDAPSNPFRDIPVARLAPRTRGNLEQLGPGHESVAALIDRLAAVPAWDSPPVWVHGDLHAANLLVADGRICAVLDFGDLTAGDPAVDLAVAWMLFDADDRRRFRIAAGGGAPVDDATWDRARLWGLHLGLIFLLHSEDSEQFSRLGARLFRAVTSEDAG

SEQ ID NO: 13 - protein, 260 aa, Propionibacterium acidipropionici
MTRTMTLPDGRTMAWEEYGAADGRPVLFLHGTPGGRLSAAKYEPFALARGLRLVAPDRPGYGLSTARPGMTLSDYAEALLDLCWWRGWGPVPVVAGSAGAAYALALGAAASEMVTGVSIFSGIAPMTDDEARTLIPVNQQLRCAVDDPAELQRLVGQVRDAILAGTLKGLPEDPALTDALKPGADGMVADYRNVFGEWGPDPVAVRVPVLWIHGTDDVNAPISAARRLASQLPEARFEEVSGGVHAPSAETLERVFDATP

SEQ ID NO: 14 - protein, 249 aa, Propionibacterium acidipropionici
MQYTLIDGLQKLLSKALSVLAAWLLVRIIDHGGFRDLALSVGWSRGLMGSLIALIVGLVVSGIAILAGDGIGLLSNTGFGESFHEQSVTLLILSIVLSIVFAAIYAVGMNTLWFGYLLRSLSGRPVIGVVVVSLVPALVSLLPAPTSRFDPSILALMAQSTPEAVGVGLASAVMVLALRSVWPSVGIAVGARLISLVSPGAMTSPSAATAVSQSAITGALFAVAALVTALLMGRHRWQQVAHVGPFATT

SEQ ID NO: 15 - protein, 396 aa, Propionibacterium acidipropionici
MEPMGVGVIGTGDISDVYLANLNKYPGFVRLVACQNRTRAKAERQAARYGIGRVHDTVEELLADPEVKLVLNLTTPEAHAPINMAALSAGRHVYSEKPLATSLEDARRTLDHAHDLGLHLGCAPDTWLGGRAQTMRDLIDSGEIGEVTAGVATIVYPGLEWFHPSPFQSYRADVGPLADIGIYYVSMLVALLGPVRQVAAMGKKTFDERTAHYGPIAGRPIPVEVETHVSASLEFEQGAVVTLLVSTDVPDSQLPRMELYGTRGTLCMPETEPMAGPNTFGGPLWIRTLDDARYKDIPRPAPTPWTEAENRHRFNETDFGADPSVPRINSRGIGLVDEVLAIAEGRPMRCSGDLACHVLDVIESIYASSRQRRFVEVASSCRRPAPLPADFPGPQV

SEQ ID NO: 16 - protein, 311 aa, Propionibacterium acidipropionici
MALNEVTFPSHDGRDQIHGWIYSPVRPVRGIVQIAHGLGEHSRRYLHMITTLLDAGFVVAADDHAGHGATAMASGVWQDTGPHGVDTVLIDERTLHDLAVELHPDLPFVFFGHSWGSMIARGYASRYPDDLSALVLCGIAAGMHGIEETLDRDALAAAIAEGDGSGPDTGFQDQMFDGFTSRCGPDAGPTAWVAADPQVVADHGIDPLNNFGAPMSLRFVRDFARLYDEVNDAAWPGTVPATVPVLILAGEQDPVANYGEGALTVANQLWDTGHEVETRIYTGVRHEVHNEPATRAQVEADLLAFVERVTG

SEQ ID NO: 17 - protein, 488 aa, Propionibacterium acidipropionici
MSSPSKTSASPQATQGGSAPAPQMKGRHLVMMSLGSAIGTGLFLGSGKGIAAAGPSVLVAYVVAGLVVIAIMRMLGEMVAAHPDSGAFSVYTARAMGPAAGFAMGWVWWVELAVVVAAEGTAAAQIFLAVWPIAPDWLLTLIFMVALTAINLFGVDKFGEFEFWFALIKVAAVVAFLVIGVLLLCGVFPAPAPGLSNFLHHGGFMPNGWGGVATGLLIVIFAFGGIEIVAVAAAETENPRKHVGKAINTIIWRILVFYMGSVAIMVFALPWDDPKLASSPFVAVLDLAKIPGADAVLTLIIVLAVLSSLNANLYGDSRMLGSLAERGLAPKAMTGKNRRNVPVAAVLSSVAFGYVCVVLTYIWGAKVLDVLLNVVGSVIIVTYLFTIASQIILRRRAEKTGEELPFRMWGYPYLSWLTLAVLIGIIGLGMTDAGVRGQILATFGLTVVLFVIGIVRTRRLGQDPFKPVVTPGDRLVDADPAAEPAGTD

SEQ ID NO: 18 - protein, 520 aa, Propionibacterium acidipropionici
MAPRPTSTDAPDDSAASDEEEVVELGTGRRITEPLRAPTAGIPAHAPRVVEMGAETVPLRAPGVAALVEELGLTASMTHPRPGPALLSSRRGAVPMPDGVTPTGPTRLLPTVRSQILSPSGLLRAAAEPITGRRHIDGDVSVGEFIETRFGPQVARAVVDPLLGAIHAADINRFSLAAAAPALVETAAEGDSMLLGTLGREARRAAGWARGLPLRGYHRMIRMMGLQEEDLDRQAPAPSLASWPRGTATLADRLAASVRARGTLLLNTRATRLTPPNDGGTAWRVGVEGLDGARELTADAVVVATGSASAAGMLADVSPRAAEILAGLRAVSVATVILDLPLDETLAAHPLSGAATWFIGSGWSPLIRQVTNLSHKWPTTLGGDRLVLRVSAGRDGGRPLDAMTDDDLARAVVAELRRLGLPVAAPAEAVTVPSKDARRRMCTLVARFPNAMPQPAPGHRGRMESLAAALSEVPGLGLGGCATDGAGVGTAILAGRRLARQISAFLDRDPRDPREQGGTL

SEQ ID NO: 19 - protein, 256 aa, Propionibacterium acidipropionici
MLVSEVMSQQTPMSRVVGPWTEWMGRWPTPDDLAEEEAGAAVAAWGRLGYPRRALRLHAAAVAIAERFDGVVPSTYAELIELPGIGDYTAAAVVSFAFGGRAAVLDTNVRRVLARVETGVANCGSATSRADRDLAAKWLPESDDDAARWAVSSMELGALVCVARAPLCESCPVAGHCRWLEAGKPTDGAPVRRGQAWKGTDRQCRGVILDLVRNSAGGVEVEVALEAWPKRHQAEKCLGTLLDDGLIHREGSVLRL

SEQ ID NO: 20 - protein, 627 aa, Propionibacterium acidipropionici
MTVVLIIVIAALFIVAAAELLADRTGIAAPILLLLLGAGVALIPGMPEVEVEPELVLMIILPPLLYSSAVNMPVADFRRNLAPISVLAVALVAVSAAVIGFIVNQMVPGIGIAACVALGAIVSPTDAVATSIVKKAGVSRRLVTVLDGEGLINDASALVILSSAVGAMFAEISAGEVILDFVLAVVVAVVVGWLVGHAMIWIRARIHEATPDTVLSMATPFLAFLPAEHLHGSGLVAAVAAGLVASHQGPRVLTPTQRMSSRTTWRSLMLILESAVFLLMGLELTAVVEDMEAESFGWKLAVAVAAVALVMTMVLRTVVVTPLLMWVTRRSKRRSKHRSYLEKASQKVADALESDEEITIRGNTIDADHAARFRHRIVRTISDLDYYIKHPLGPREGSVMIWAGMRGAITLAAAQTLPLDTPHRSFLVFVAFLVAAASLLIQGSTLGLVVKVAKPATSEGVDPDEQAEIRKLMHRAARKVPVPAPMRRLLARTGHQESEDVEENRAQAAAVALAWRQFAALRDRGVESEAPAEAGPERLGETAPIPRIVGKQLPGEDARAKVLASPEHRRRAAEISRQYALRLIVAQRKVLLDANDAGRFSPEAVSSALDTLDADQLSLEARGTSLD

SEQ ID NO: 21 - skipped

SEQ ID NO: 22 - protein, 540 aa, Propionibacterium acidipropionici
MSILRTKSVEQSLRDTEDPEHQLKKSLSWVELTMFGIGVVIGAGIFTMTGRVAHSMTGPSIIISFIVAAIACGLAAMCYAEFASTVPVAGSAYTFSYASMGEIFAWIIGWDLFLELFLASSVVAQGWSAYLAVFLSQLGIDLPPQIVSGGRFDLLAFGLIMVLGMLLIGGIKESVRVNTVLVAIKLFIVMFVIFAGIGYVKASNFTPFVPDKQPVESTGGLTQPLLQWFTGSGQTAFGVSGIVAGAALVFFAYIGFDVVATTAEEAKNPKRDVPLGILGSLVVCTILYIAISLVLIGMVPYDQLDPSASLAKAFTTVGKPWMAIIISAGAVAGLTTVVLTMMIGATRVIFAMSRDGLLPEGLSHVHPKTRTPYRITLIIMLADGLLAALVPPGILDEMVNIGTLLAFVMVSVGIIVLRRKRPDLPRAFRVPWVPVVPIVSAIICLYLMLNLSIETWMRFLIWMVIGIVVYFTYSKNHSRLAHGSELTADINAEITNVMGHQYDARRGSRRAGSPAAQASSAGSAEPADAAPELSDPPKES

SEQ ID NO: 23 - protein, 674 aa, Propionibacterium acidipropionici
MRDAMRATVPALALAVALSACSGSGQSRGSASASGDGVASLTETKVTPLVSSIGSRDGADLKPVRLADGLTPPTNRWFSGMAYGSTAQPVFPLPLSFSLLGSGFALGLPDIKTSDRTIMGGNRPAVQIGAGADSWKITRYDEMSVTLTGSAGGTEIGTVTIARGSPFVTFRASGRRTLTTNLPFTGSGSPWSLQAGEDRYDLTGSKGVSVSGGAVTVPDGGHVTLYPEPEGGDAAALARLAASPLRSTASSYRLSGSTATTRLAYSTDDGSPTAIAALPHQQAGLATGQHCDLGSYRSVLGTMKLCRGTALSWDTKTRPATAQLDLSGLADDQRAALRTQVDADVRALKPYPADTYFGGKALYRDAQLYTLAKQVGASSSATTLKSRIVEQLTKWADPSGCGSRTSLCFYYDRSNKGMVGLTHSFGSEQFNDHHFHYGYFLYAAGVMAADDPSLVKRWKPVMTLLAADIASPTDTGTFPQRRTFDPYSSHSWASGVSPFGDGNNQESASEAVNAWVGLGVWARAAGDPQLAAEGTWMQALESDSQLAYWTNFDTDDPVYKGFGHSITPLVWGGKRDYATWFSPEPAAALAILLIPMNPASGYLGTDPKRVATNLKEAMGTRGYRQTYGDLLLLYSALQGSSQRDAAVKQVASLTSIDDSLTRSYILAYLYALKF

SEQ ID NO: 24 - protein, 425 aa, Propionibacterium acidipropionici
MTSPARAHVDDVISFVESSPTSYHAAAELARRLEEAGFERLDETADWSGAASVEGRRFVVRDGAVIAWATPETIGPRAGFRIVGSHTDSPSFKLKPHATFTNLGWQQVGMEVYGGGLLNSWLDRDLGLAGRLVTLDGETHLVRTGPILRISQLAPHLDRTVNQDLTLDRQRHLMPILSVGRPDLDVEDLLCEEAGIDRSRLGFHDILAYPTERPAVIGPAGEFLASSRMDNLSSVHSSIAAMVDVEVGEDIAVMACFDHEEVGSSTRSGACGPFLEDVLVRIADGLGRRGDAYRAMIARSTCISSDAGHGVHPNYPEKFDPANHPLLGQGPLLKINANQRYATDGVGGALWQRVCRAADVPTQAFVSNNSVPCGSTIGPLTATRLGMLTVDVGLPLMSMHSTRELAGVADLSSLSTALGAFWAGA

SEQ ID NO: 25 - protein, 713 aa, Propionibacterium acidipropionici
MPAPRTAFLDLSALAPAKTRPALIDADLARFTAIAEAIDAQLTTLTAQRRDALSDTARTGRAAADRDQEVRRINSRIRILRDVGPQICLGRMDRTGGEPVYIGRIGLSDDADRRLLVDWRTPAARPFFAATVADPMGLAGRRRFRWRDGRVIDYWDEALIPDAGTDPATMDAESAFIASLAASRSPRMLDVLATIRADQDAAVRAEARRPLIVEGGPGTGKTVVALHRAAYLLHADPTLNNRGGGVLLIGPHPGYLAYTADVLPDLGEDGARTATVADLLPQGPDARPEPDTRVAALKLDARMAGAVEPAVALYEEPPTDTLTVDTPWGEVAVTAAAWTEAFSAAEPGSVHNEARDRIWHDLIDILVDHAQSLDAPEDRLRRALYGDEELREAFSRAWPILNPEELVADLWEVPAYLRRCAPWLTPDEAALLRRGPAHPWTTADLPLLDAATRRLGDRRAGAAARRRQAVLAEQRSYMDDVVTHILDADDDPDSSLAMLRGADLRQVLLDTDALDDGTTDPLAGPFAHIIVDEAQELADAQWQMLIRRCPSLSFTIVGDRAQARDGFPESWEERLGRLGFHDMSRVTLSVNYRTPSEVMEAAEPVIHAALPDAAVPTSVRSSGLPVRHGRIAHLDRIVAEWLDENPEGIAAVIGAPGFDGGPRVRALSPADVKGLEFDLVVIVDPEHFGDGITGAVDRYVAMTRTTSQLVILR

SEQ ID NO: 26 - protein, 436 aa, Propionibacterium acidipropionici
MTHSENLTDISRTIDDFDSITDANKAVVDVMTAGQPVLVDVARAHTLIPELDTGEKVLLHAGPPIDFEHMPETIKGACIGAALFEGWASDEDAARRVVAEQVRLIPCHHVGAVGPMGGITSAHMALMKVVNTTYGNVSYSTLNEGIGKVLRFGGYDAEVIDRLGWMRDVLGPALSSALATTDGGYPLAPVMARALTMGDEMHQRNIAASALFAKDMAPLLARAGLPGDTVAEVSDFLGRTDQFFLNVAMAASKACADPARQVRAGSVVTAMCRNGYEFGIRVSGLGDRWFTAPVNTPSGLFFTGYDQSQAAPDMGDSAIMETFGLGGMSIVAAPGVTPFLGAGGFSEALATTEEMAEVVTAHNPNMPIPTWNFQGAPTGIDIRLVVQTGITPIINSGIASKHPGVGQIGAGTVRAPMGCFTRAVEALAEVYGVDVS

SEQ ID NO: 27 - protein, 419 aa, Propionibacterium acidipropionici
MVCPSTLFLVTNSYPLGTGEDFIENEIGDLAERFGRVVVVAVQTRPGDVITRPVPGNVEVIRAGGPRPAGRAALLAAARGLAHLPRGSWNRDTLRDPRRLGLEAMFEEHARDTEADLLAQLPALGLRPGSHAVVYSYWFLDTARVAMLLAADLRARGVVVDRLVSRAHGYDLYPERAPYGHLPQRERLVAAFDAVCPVSEQGTRTLRSGWPGYAGKIGTHHLGTVGPGSPADCSREPFHIVSCAYLVPVKRMTRMPGVLAELRGRGVDARWTHLGGGPESEDVLKAARDAGVDEQVDLQGHLAHEKILETERGLRPSCLINLSSSEGLPVSMMEAASLGIPLIGTDVGGVREIITDRVNGRLINPDFTDSQAADTLQWLADLPTDDYRSVCEASRRIWQSDYDQAVVYPRFCTEVLGAD

SEQ ID NO: 28 - protein, 160 aa, Propionibacterium acidipropionici
MRRYTITVAGTTHEIEVEETSATQFQVRVDGQQVEVELTAHQDVGDQPVVPQIDPRHESAPKAPRVPQQVRTRATGSPSPALTGGADLAYSMTAPMPGVIASVDAGPGDEVAKGQTVLVLEAMKMKNELHASRSGVIAEVLVAEGDQVKYGQTLLCFEKA

SEQ ID NO: 29 - protein, 731 aa, Propionibacterium acidipropionici
MGRFFKNLAFTTLVLVLVLAIAAGVGAIVFYNRTNLPDPNKDFQTNTSFIYFNDSKTKLGSLSVQNRQTIGYEQMPKSIKEAAISAENRTFWSDQGISIGGIVRAAWTIARGGEMQGGSTITQQYIKILYLSQDRTMQRKLKELVLAVKMGKQVPKEDILAGYLNTIYFGRGAYGIQAAAKSYFNVDASKLTVPQSAVLASILNNPTLFDPSGGAKARERLLNRYRYVLDGMLEAGNITQAEHDEYSRKLPAFPEVPINNRWGGTNGYLLKMVQNELLDDGFTDSQINGGGLKVTTTFDPAAQKAAVATGQKYKKLAGSNAGKNGAKNLHPAIASVKVGTGEVLALYGGDDYITSTRSWALQARPAASTFKTYAVIAGMRNGFSLKSKLNGDTFTPQGDSVPIRNEFSEQYGDVTLQKATEDSINTAFVDMMTQIDNGPQAMLKAANDAGVPKGSGWDLNNRMPLGVAEVSPLDQATGYATIANEGKYVPSHVVAKVTDSSGKTLYTAKTTGKQTIQKDIAHDTTYALENVVNEGTGSAVSNLGYPVAGKTGTNGVKDDITSAWFVAYTRQISTAVMYVAGDGGNEDLDPYAAEGDSTFFGGTYPARTWASYMKVAMKGLPSQDFPKPDKVNLSGKHYGNTQRETLRTPTPTPTPTPSETRTPTPEPTQSSTPSRPEPTQTSSEPEPTDTPSQPEPTDTPSRPLPSLPKPTLPGIPGPSDGDDGDNGATGR

(19) United States
(12) Patent Application Publication
(10) Pub. No.: US 2024/0327879 A1
(43) Pub. Date: Oct. 3, 2024

(54) METHODS FOR ORGANIC ACID PRODUCTION

(71) Applicant: S&P Ingredient Development, LLC, Saint Louis Park, MN (US)

(72) Inventors: Paul Blum, Monterey, CA (US); Sambasiva Rao Chigurupati, Omaha, NE (US); Derrick Jermaine White, Lincoln, NE (US)

(21) Appl. No.: 18/741,273

(22) Filed: Jun. 12, 2024

Related U.S. Application Data
(62) Division of application No. 18/188,061, filed on Mar. 22, 2023, now Pat. No. 12,054,763.
Application No. 18/188,061 is a division of application No. 17/073,977, filed on Oct. 19, 2020, now Pat. No. 11,613,769.
Application No. 17/073,977 is a continuation of application No. 16/443,554, filed on Jun. 17, 2019, now Pat. No. 10,808,266.
(60) Provisional application No. 62/686,463, filed on Jun. 18, 2018.

(51) Int. Cl. C12P 7/52; C12N 1/20; C12R 1/02; C12R 1/145; C12R 1/225

(52) U.S. Cl. CPC C12P 7/52; C12N 1/205; C12R 2001/02; C12R 2001/145; C12R 2001/225

(57) ABSTRACT

Microbial cell lines suitable for industrial-scale production of organic acids and methods of making and isolating such cell lines, and using the cell lines for production of organic acids.

### CLAIM OF PRIORITY

This application is a divisional application of and claims priority under 35 U.S.C. § 120 to U.S. application Ser. No. 17/073,977, filed on Oct. 19, 2020, which is a continuation application of and claims priority under 35 U.S.C. § 120 to U.S. application Ser. No. 16/443,554, filed on Jun. 17, 2019, now U.S. Pat. No. 10,808,266, which in turn claims the benefit under 35 USC § 119 (e) to U.S. Patent Application Ser. No. 62/686,463, filed on Jun. 18, 2018, the entire contents of each of which are incorporated herein by reference.

### SEQUENCE LISTING

This application contains a Sequence Listing that has been submitted electronically as an XML file named “37942-0022003_SL_ST26.XML.” The XML file, created on Feb. 13, 2023, is 37,383 bytes in size. The material in the XML file is hereby incorporated by reference in its entirety.

### TECHNICAL FIELD

The disclosure generally relates to microbial cell lines that overproduce organic acid and methods of making the same.

### BACKGROUND

Organic acids refer to carbon-containing compounds having acidic properties. Examples of organic acids include acetic acid, citric acid, gluconic acid, lactic acid, propionic acid, among many others. Because they are fully degradable, organic acids can be used in the production of biodegradable polymers. They also have other important industrial applications, including as food additives.

### SUMMARY

The disclosure provides microbial cell lines suitable for industrial-scale production of organic acids and methods of making and isolating such cell lines.

In one aspect, a method of making and isolating a microbial cell line is provided, where the isolated microbial cell line overproduces an organic acid compared to the parental microbial cell line. The method uses serial passage of a parental strain in pH-controlled culture media supplemented with the organic acid, preferably in non-immobilized culture, where the pH is controlled at a value above the pKa value of the organic acid. In some embodiments, the pH is preferably in the range between about 5.5 and about 7.5, more preferably at or near neutral, between about 6.0 and about 7.0, and most preferably at about 7.0. In some embodiments, the culture media is solidified. In some embodiments, the culture medium is supplemented with the organic acid in an amount sufficient to inhibit normal microbial cell growth, e.g., to reduce doubling rate or growth rate, e.g., by at least 5%, 10%, 20%, 30%, 40%, 50%, or more. In some embodiments, the organic acid is supplemented at a progressively increasing amount in successive iterations of the serial passage. In some embodiments, the organic acid is supplemented at the same amount in successive iterations of the serial passage. In some embodiments, the organic acid is propionic acid, lactic acid, acetic acid, or butyric acid. In some embodiments, the organic acid is propionic acid, e.g., the culture media is supplemented with about 1.0%-3.0% of propionic acid, e.g., about 3.0% of propionic acid. In some embodiments, the parental cell line is a wild-type organism. In some embodiments, the parental cell line is a microbial cell line is derived from unicellular microbes.

In another aspect, a microbial cell line that overproduces an organic acid is provided, where the microbial cell line is made and isolated using serial passage in pH-controlled culture media supplemented with the organic acid, where the pH is controlled at a value above the pKa value of the organic acid, preferably in the range between about 5.5 and about 7.5, more preferably at or near neutral, between about 6.0 and about 7.0, and most preferably at about 7.0.

In another aspect, a microbial cell line that overproduces an organic acid is provided, where the microbial cell line has mutations that primarily alter, directly or indirectly, the structure, composition, and/or function of the cellular envelope. Preferably, the microbial cell line includes at least 2 genome mutations identified in Table 3 or analogous mutations. More preferably, the microbial cell line includes all of the genome mutations identified in Table 3 or homologous mutations. In one embodiment, the microbial cell line includes mutations in at least 2 genes identified in Table 3 or analogous mutations thereto. In another embodiment, the microbial cell line includes mutations in all of the genes identified in Table 3 or their homologs (e.g., homolgous genes in another species described herein). In another embodiment, the microbial cell line includes a mutation in O-antigen ligase domain-containing protein. In another embodiment, the microbial cell line includes a mutation in M18 family aminopeptidase. In another embodiment, the microbial cell line includes a mutation in amino acid permease. In another embodiment, the microbial cell line includes a mutation in adenine glycosylase.

The microbe can be any microbe that produces an organic acid. In one embodiment, the microbe is from the genus Propionibacterium (Acidipropionibacterium), and more preferably the species P. acidipropionici. In another embodiment, the microbe is from the genus Lactobacillus, and more preferably the species L. acidophilus. In another embodiment, the microbe is from the genus Acetobacter. In another embodiment, the microbe is from the genus Gluconobacter. In another embodiment, the microbe is from the genus Clostridium, and more preferably the species C. butyricum. In some embodiments, the organic acid is propionic acid. In some embodiments, the organic acid is lactic acid. In some embodiments, the organic acid is acetic acid. In some embodiments, the organic acid is butyric acid.

Also provided herein are methods of producing organic acids using the methods and microbes described herein.

### BRIEF DESCRIPTION OF THE DRAWINGS

FIG. 1 shows growth of wild-type and mutant P. acidipropionici on solid buffered medium with or without addition of 1.0% PA, showing the phenotype of Strain 3-1.

FIG. 2 shows production of PA by a mutant strain of P. acidipropionici (Strain 3-1) relative to wild-type in bioreactors using bleached American Beauty Cake Flour (WFM as bolus). 1:200 inoculation, 1 L working culture, 5% (w/v) glucose equivalent WFM, 30° C., pH 7 (NaOH, 5M).

### DETAILED DESCRIPTION

The most common organic acids are carboxylic acids, whose acidity is associated with the carboxyl group (—COOH). They are generally weak acids with pKa values between about 4-5. Propionic acid (“PA”), for example, is a carboxylic acid with the chemical formula C₂H₅COOH or C₃H₆O₂. It is a colorless, oily, and pungent (think Swiss cheese and sweat) liquid and has physical properties between those of the smaller carboxylic, formic, and acetic acids, and the larger fatty acids. It has a molecular weight of about 74.1 g/mol, and a pKa of about 4.9, which means in a solution having a pH of about 4.9, half of the PA is in the protonated (or undissociated), uncharged state (C₂H₅COOH), while the other half is in the deprotonated (or dissociated), negatively charged state (C₂H₅COO⁻), known as propionate or propanoate ion, which can form salt or ester compounds. As the pH decreases (becoming more acidic), more PA is in the protonated, uncharged state; when the pH increases (becoming more basic), more PA is in the deprotonated, negatively charged state.

Because PA inhibits the growth of mold and some bacteria at levels between 0.1 and 1% (w/v), PA and its salts are used as a preservative in both animal feed and human food (such as baked goods). In the United States, PA is “generally recognized as safe,” or GRAS, by the Food and Drug Administration when used as a food additive. It is also approved for use as a food additive in Australia, New Zealand, and the EU. In addition, PA is an important intermediate in the synthesis of other chemicals, such as cellulose-derived plastics, pesticides, fruit flavors, perfume bases, and pharmaceuticals.

While they are widely distributed in nature, commercial production of organic acids has generally relied on chemical synthesis because it is more economically competitive. For example, PA is currently commercially produced almost exclusively through petrochemical processes. As prices for crude oil and petrochemicals increase, along with the rapid development in the biotechnology field, the economic gap between manufacturing costs of PA via chemical synthesis and via microbial fermentation is narrowing. Coupled with growing concerns about energy shortages and environmental pollution, there has been an increasing interest in commercial-scale biosynthesis of organic acids such as PA from renewable resources.

Microbial production of organic acids by fermentation has been known and used for centuries. For example, Aspergillus niger and Yarrowia lipolytica have been used to produce citric acid; Lactobacillus has been used to produce lactic acid; Clostridium has been used to produce acetic acid; Aspergillus niger and Gluconobacter have been used to produce gluconic acid.

Propionibacterium is the microorganism most often used in the production of PA (as well as vitamin B₁₂ and Swiss cheese). Propionibacterium is a gram-positive, non-motile, non-spore forming, rod-shaped, anaerobic genus of bacteria that includes the species P. freudenreichii, P. acidifaciens, P. cyclohexanicum, P. australiense, P. acidipropionici, P. jensenii, P. thoenii, P. microaerophilum, P. olivae, P. damnosum, P. propionicum, P. acnes, P. avidum, P. granulosum, P. humerusii, and P. lymphophilum. For industrial PA production, the most commonly used strain is P. acidipropionici. (A proposal has been made to reclassify the species within the genus Propionibacterium into three novel genera: Acidipropionibacterium, Cutibacterium, and Pseudopropionibacterium (Scholz & Kilian 2016). However, Propionibacterium acidipropionci and Acidipropionibacterium acidipropionici are still used somewhat interchangeably.) The optimal pH and temperature for Propionibacterium cell growth are about 6.0-7.0 and about 30-37° C., respectively (Ahmadi et al. 2017). Cell growth is inhibited in pH less than about 5.0, although fermenters started at neutral pH can reach pH 4.4 (Rehberger and Glatz 1998). Ahmadi et al. provides an overview of PA production on several carbon sources by various species of Propionibacterium as reported in the literature (Ahmadi et al. 2017) and is incorporated herein by reference.

PA can also be produced by other anaerobic bacteria, such as certain species of Anaerovibrio, Bacteroide, Clostridium, Fusobacterium, Megasphaera, Propionispira, Selenomonas, and Veillonella.

There are a number of fermentation pathways that convert carbon sources to PA through a series of enzymatic reactions. The primary fermentation pathway involved in PA production, especially in propionibacteria, is known as the Wood-Werkman cycle, which produces propionate from pyruvate, the terminal product from glycolysis, and involves many intermediates, including oxaloacetate, malate, fumarate, succinate, succinyl-CoA, methylmalonyl-CoA, and propionyl-CoA, and many enzymes, including oxaloacetate transcarboxylase, biotin-dependent carboxytransferase, CoA transferase, fumarate hydrolase, lactate dehydrogenase, coenzyme B₁₂-dependent methylmalonyl-CoA mutase, malate dehydrogenase, and succinate dehydrogenase.

While most pyruvate is converted to PA/propionate during fermentation, some is converted to acetate. The acetate formation pathway involves intermediates acetyl CoA and acetyl phosphate, and enzymes pyruvate dehydrogenase complex, phosphotransacetylase, and acetate kinase.

A number of carbon sources have been used for microbial PA production, including glucose, fructose, maltose, sucrose, xylose, lactose, glycerol, lactate, flour hydrolysate, molasses, whey, and a combination thereof. A number of culture systems such as batch, fed-batch, and continuous fermentation have been used.

However, for commercial-scale microbial production of organic acids to be economically viable, the fermentation process must be able to convert carbon sources at a high yield (amount of organic acid production from carbon source, typically measured in g/g) and high productivity (rate of organic acid production, typically measured in g/L·h).

Various fermentation technologies, including fed-batch, continuous culture, multi-stage, cell immobilization, and extractive fermentation systems, have been explored to increase the yield of organic acid production. However, the modest increase in yield and productivity often comes is offset by a significant increase in production cost.

For example, coculture methods have been used to produce PA using whey as feedstock (WO 85/04901; EP 0141642 A1). WO 85/04901 describes the use of Lactobacillus casei subspecies rhamnosus in the presence of Veillonella cricetid to interconvert lactate to propionate via a two-stage fermentation process. In the first stage, carbohydrates are converted to lactic acid by L. casei; in the second stage, lactic acid is fermented to PA by V. cricetid. (The genera Lactobacillus and Veillonella both belong to the phylum Firmicutes, whereas the genus Propionibacterium belongs to the phylum Actinobacteria.) EP 0141642 also describes the use of a mixed culture of lactic acid-producing bacteria (L. casei) and PA-producing bacteria (P. shermanii) to maximize the fermentation yield. The coculture systems of WO 85/04901 and EP 0141642 are reported to be very productive in terms of PA production from lactose, with final yields ranging from 20-100 g/L. However, such coculture systems have considerable implications for process parameters. For example, they suffer from a lack of control over the growth and metabolic activity of each member of the system, which can lead to failure of either member to grow or to contribute to formation of the desired product. A lack of reproducibility is common with coculture systems.

One major problem associated with microbial production of organic acids is the strong inhibitory effect of the end product on cell growth and the fermentation process, leading to low production yield and productivity. Acid tolerance was assumed to be crucial to improving the yield and productivity of PA-producing strains (Rehberger and Glatz 1998). The elevated inhibitory effect of PA at pH 4.5-5.0 as compared to lactic acid was attributed to the fact that at this pH range, about half of PA (which has a pKa of about 4.9) would be present in the undissociated, protonated, and uncharged form, whereas lactic acid (which has a pKa of about 3.1) would mostly be in the dissociated, deprotonated, and charged form. It was assumed that because the undissociated acid could penetrate the cell wall and membrane more easily, more PA than lactic acid could get into the cell and exert its inhibitory effect. Enhancement of acid tolerance was thus thought to be an effective strategy to alleviate end-product inhibition and improve PA production. Accordingly, attempts have been made to create “acid tolerant” mutants of propionibacteria under high PA and either uncontrolled or low pH conditions.

For example, adaptive evolution via serial passage has been used to obtain mutant P. acidipropionici with improved acid tolerance (Woskow and Glatz 1991; Zhu et al. 2010). Serial passage is a method of growing microorganisms such as bacteria in two or more iterations in artificial environments, often created in a laboratory setting, to generate spontaneous mutations in the microorganisms as they evolve over the course of the experiment to adapt to one or more new environmental conditions designed for the experiment. For example, repeatedly subjecting microbes to extreme acidic conditions will lead to spontaneous mutations that allow the microbes to adapt to or tolerate such conditions.

In prior work, to create mutations that confer acid-tolerance, the mutant P. acidipropionici strains were adapted to increasing PA concentrations by repeated and serial transfers in selection media containing increasing amounts of PA (from 0.5% to 5% (Woskow and Glatz 1991) or 1.5 g/L to 20 g/L (Zhu et al. 2010)) over a period of one year or longer. Importantly, in these experiments, pH in the selection media having increasing amounts of PA was not controlled, presumably because it was assumed that the inhibitory effects on cell growth and PA production were caused by the acidity of PA.

P. acidipropionici mutant(s) with enhanced PA production has also been obtained by immobilization and adaptation in a fibrous-bed bioreactor (Suwannakham and Yang 2005; Suwannakham 2005). The ability to obtain acid-tolerant mutant(s) in fibrous-bed bioreactor was attributed to the high cell density and viability maintained in the bioreactor and distinct physiology and survivability of immobilized cells as a result of their direct contact with each other and with a solid surface. The higher PA production was attributed in part to higher activity levels of oxaloacetate transcarboxylase and CoA transferase in the mutant(s). Despite the higher PA yield, in the fibrous-bed bioreactor with high cell density, cell growth is limited. Moreover, fibrous-bed bioreactors are expensive and not scalable, and their uses are limited to small-to-medium scale productions.

More recently, random mutagenesis strategies such as genome shuffling have been used to accelerate directed microbial evolution. For example, Guan et al. reported the use of genome shuffling to generate an acid-tolerant mutant P. acidipropionici strain (Guan et al. 2012). To obtain the strain, four successive rounds of genome shuffling via protoplast fusion were performed, and the acid-tolerant strain was selected using media supplemented with increasing amounts of PA (from 5 to 20 g/L). Again, pH in the selection media having increasing amounts of PA was not controlled, presumably because it was assumed that the inhibitory effects on cell growth and PA production were caused by the acidity of PA.

Subsequent analyses identified 24 proteins that significantly differed between the parental and shuffled strains (Guan et al. 2014). The detected proteins were reported to fall into four broad functional classes: cellular metabolism and energy production; DNA replication, RNA synthesis, and translation; posttranslational modification, protein folding, and chaperones; and hypothetical proteins of unknown function.

In another study, genome shuffling was used to generate acid-tolerant mutant P. acidipropionici, P. intermedium, and P. jensenii strains (WO 2017/055932 A2). Three successive rounds of genome shuffling were performed for each set of strains, each followed by selection of colonies from the acidic (pH 3) side of pH/PA gradient plates prepared using agar culture media supplemented with 5 g/L of PA at either pH 3 or pH 6.5. Final individual recombinants were randomly selected after serial dilutions in culture media plates and screened in a 96 well plate containing 100 μl of culture media at pH 5 and 25 g/L of PA. The mutant strains were reported to have enhanced yields of PA relative to native Propionibacterium and other known derivative strains. Genomic analyses of one of the mutant P. acidipropionici strains identified a number of modified genes, including those encoding the ABC polar amino acid transporter, the Cytochrome C biogenesis protein, the ABC multiple sugar transporter, the large subunit of ribosomal RNA, the long chain acyl-CoA synthetase, and the cation diffusion facilitator. In addition, an extra copy of the whole ribosomal RNA gene and an extra copy of the arginine deiminase regulon (ArgR) with a point mutation were found in the mutant strain.

Targeted metabolic engineering of propionibacteria has also been used to increase PA production. These studies generally target enzymes involved in pyruvate metabolism pathways to, for example, either inhibit the acetate formation pathway or enhance the PA formation pathway. For example, Yang and Suwannakham created engineered P. acidipropionici strains with genes encoding acetate kinase (which catalyzes conversion of acetyl phosphate into acetate) and/or phosphotransacetylase (which catalyzes conversion of acetyl CoA into acetyl phosphate) knocked out, with the goal of eliminating or reducing acetate formation and thereby enhancing PA production (US 2011/0151529 A1; Suwannakham 2005).

Yang et al. created engineered P. acidipropionici and P. freudenreichii subsp. shermanii strains transformed with propionyl-CoA: succinate CoA transferase genes to increase PA production by overexpression propionyl-CoA: succinate CoA transferase, which catalyzes conversion of propionyl CoA into propionate (WO 2012/064883 A2). The resulting strains were reported to have increased PA production and resistance to PA, as well as resistance to acidic pH in general. The increased CoA transferase activity is believed to increase carbon flux through the PA formation pathway over the acetate formation pathway.

The table below describes a list of genes that have been manipulated using recombinant DNA. These genes constitute conventional genetic targets where regulatory mutations might be expected to increase PA yields.

TABLE 1

| Gene(s) | Organism | Effect | Reference |
|---|---|---|---|
| OtsA (trehalose | P. acidipropionici | Artificially | Jiang et al. 2015 |
| biosynthesis) | | over-expressed | |
| Several genes in arginine | P. jensenii | Artificially | Guan et al. 2016 |
| deaminase and glutamate | | over-expressed | |
| decarboxylase systems | | | |
| Propionyl-CoA:succinate | P. acidipropionici | Artificially | Wang et al. 2015 |
| CoA transferase | P. shermanii | over-expressed | WO 2012/064883 A2 |
| Acetate kinase | P. acidipropionici | Artificial | Suwannakham et |
| | | knock out | al. 2006 |
| | | | Suwannakham 2005 |
| | | | US 2011/0151529 A1 |
| Phosphotransacetylase | P. acidipropionici | Artificial | US 2011/0151529 A1 |
| | | knock out | |

Targeted genetic engineering in propionibacteria, however, is challenging. As an initial matter, the effect of acid alteration and stress on bacterial physiology is complex and not well understood, making it difficult to improve tolerance towards organic acids through manipulation of specific genes. Indeed, despite knowledge about the identity of the intermediates and enzymes in the Wood-Werkman pathway that form PA in propionibacteria, genetic manipulations of the genes in this pathway have not increased PA yields to a significant extent.

Moreover, the high GC content in propionibacteria makes it difficult to identify the locations of individual genes and all of the coding regions in the genome, which complicates genetic manipulation. In addition, there are only a small number of cloning vectors available for introducing recombinant DNA into propionibacteria cells, which are known to have low transformation efficiency. Selection of transformants is also complicated by the ability of propionibacteria to quickly develop spontaneous resistance to antibiotic markers.

In addition to these challenges, the use of recombinant DNA for producing microbial cell lines is incompatible with the development of an organic food ingredient such as PA. At least in the United States, PA or other organic acids produced by genetically engineered microbes cannot be labeled as “organic” or “natural preservative,” which is especially important in the food industry. Therefore, there remains a need for new microbial strains suitable for industrial-scale production of organic acids and methods of making and isolating such strains.

The toxicity of organic acids towards microbes is not well understood despite its relevance in the food and chemical industries that use fermentation for organic acid production. Despite knowledge about the identity of the intermediates and enzymes in the Wood-Werkman pathway that forms PA in propionibacteria, genetic manipulations of the genes in this pathway have not increased PA yields to a significant extent. One reason could be that these genes do not limit PA formation. Therefore, altering their sequence or expression would not change PA levels. Instead, it is argued here that other cellular targets control PA yields, but their identities could not be predicted based on current knowledge. The unknown process is what limits PA formation. Since this process is not known, the genes involved in this process cannot be predicted.

Prior efforts in creating PA-resistant bacteria through serial passage or genome shuffling have generally used media with increasing amounts of PA but either without pH control or at a pH significantly below the pKa of PA. This is based on the idea that toxicity, and therefore resistance, arises from the concentration of the organic acid. However, this approach does not consider the mechanism of organic acid uptake by the cell that involves the transporter system, which depends on the nature of the transporter and the membrane or envelope in which it is located.

Organic acids are weak acids with pKa values generally between about 4-5. The relationship between pH and pKa is described by the Henderson-Hasselbalch equation:

$$pH = pKa + {log}_{10} ( [ {A}^{-} ] / [ HA ] )$$

wherein [HA] is the concentration of the protonated, undissociated, and uncharged weak acid, and [A⁻] is the concentration of the deprotonated, dissociated, and negatively charged conjugate base. In a typical fermentation process, the pH of the microbial culture when the organic acid reaches maximum concentration is approximately at the pKa of the organic acid without the use of a buffering agent. A solution having a pH of about 4-5 is not that acidic relative to the known pH tolerance of organic acid producing bacteria. Most of these bacteria do grow at pH values in this range, although the optimum pH for cell growth is typically about 6-7.

Intracellular transport of organic acids can be achieved through diffusion or through the action of membrane transport protein systems depending on whether the organic acids are charged or uncharged. When organic acids are not deprotonated or dissociated, they are uncharged. In this state, they can diffuse across the cellular membranes without reliance on transport systems. Charged molecules, however, always require a transport system to be translocated across membranes.

At a pH value that equals its pKa value, half of the organic acid is in the protonated (or undissociated), uncharged form, while the other half is in the deprotonated (or dissociated), negatively charged form. At pH values below their pKa values, organic acids would mostly be uncharged because their carboxyl groups would be protonated. At pH values above their pKa values, organic acids would mostly be unprotonated or dissociated and therefore negatively charged.

At high concentrations of the organic acid, the pH is relatively low, and the organic acid would mostly be in the uncharged state and could diffuse into the cell in its acid form. This is the basis for prior efforts to isolate organic acid resistant microbes either without pH control or at a pH significantly below the pKa of the organic acid. The approach in theory would generate cell lines with mutations that produced resistance due to diffusion-based organic acid cell entry. It was assumed that the uncharged organic acid would diffuse through the cell membrane into the cytoplasm and release protons due to the relatively alkaline pH inside the cell; the increase in intracellular acidity would inhibit cell growth and organic acid formation. In other words, it was assumed that organic acids in their uncharged state limited their own production. Despite the published literature and patents, in our experience, this approach does not generate resistant microbes effectively, and may require years of passage to work.

We hypothesized that it was not the acidity of the organic acid that was toxic, as previously assumed by others. Rather, it was the deprotonated, negatively charged form or the neutral salt of the organic acid (propionate) that was toxic, and would be more effective as a selection agent to recover spontaneous resistance mutations.

Unlike prior efforts, we hypothesized that the use of pH control at a value above the pKa value of the organic acids to be produced, and preferable at least 1 unit above, would ensure that most of the organic acids remain in a charged and deprotonated form. In this form, they would remain dependent on protein transport systems for intracellular uptake. This would avoid recovery of cell lines with mutations that produced resistance due to diffusion-based organic acid cell entry, if such mutations could be discovered.

Specifically, the process used was serial passage of the starting microbial cell line (usually but not necessarily a wild-type) in free-cell (i.e. non-immobilized or planktonic) culture in a bacteriologic culture medium supplemented with organic acid of interest in an amount that is sufficient to inhibit normal microbial growth (either in progressively increasing amounts or the same amount for all passages) under conditions of continued pH control at a specific pH that is above the pKa value of the organic acid. The pH is controlled at a value above the pKa value of the organic acid, preferably in the range between about 5.5 and about 7.5, more preferably at or near neutral, between about 6.0 and about 7.0, and most preferably at about 7.0. Although the present examples describe the use of Propionibacterium, other microbes can be used that are fermentative organisms that excrete organic acids, e.g., Lactobacillus, Acetobacter, Gluconobacter, or Clostridium. The organic acid used can be, e.g., PA, lactic acid, acetic acid, or butyric acid. In some embodiments, the microbe is from the genus Propionibacterium (Acidipropionibacterium), and more preferably the species P. acidipropionici, and the organic acid is PA. In some embodiments, the microbe is from the genus Lactobacillus, and more preferably the species L. acidophilus, and the organic acid is lactic acid. In some embodiments, the microbe is from the genus Acetobacter or the genus Gluconobacter, and the organic acid is acetic acid. In some embodiments, the microbe is from the genus Clostridium, and more preferably the species C. butyricum, and the organic acid is butyric acid.

Using our method of serial passage with pH control, we were able to create and isolate a new microbial strain having increased organic acid production compared to the parental strain in less than two weeks, much faster than using the conventional serial passage method described in Woskow and Glatz 1991, which generally takes at least one year. Our method is also much less complex and more easily scalable than other random mutagenesis methods such as genome shuffling and cell immobilization in a fibrous-bed bioreactor or targeted genetic engineering. Organic acids produced by mutant cell lines created and isolated using serial passage with pH control can be labeled as “organic” or “natural preservative,” which is especially important in the food industry.

The same method of serial passage with pH control can be used to make and isolate a variety of microbes, including but not limited to Propionibacteria, Lactobacilli, Acetic acid Bacteria, and Clostridia, that overproduce a number of organic acids, including but not limited to PA, lactic acid, acetic acid, and butyric acid. All charged molecules depend on transport systems and their associated membranes/envelopes for function. Alterations in these cellular components would achieve the same outcome as described here for propionate for other organic acids.

The same selection method (i.e., using bacteriologic culture medium supplemented with organic acid of interest in an amount that is sufficient to inhibit normal microbial growth under conditions of continued pH control at a pH that is above the pKa value of the organic acid) can be used in screening microbial libraries generated from genome shuffling or other random mutagenesis methods for isolates that exhibit increased organic acid tolerance and production.

Using this pH control method, we were able to target unique mechanisms for resistance that depended on transport and/or unpredictable intracellular targets including those involved in regulation and metabolism. Genome resequencing was then used to identify the critical genes through their mutational changes that caused the genetic resistance to high concentrations of organic acids.

The resulting mutations generally affected cellular envelope functions, as shown in Table 2.

TABLE 2
ENVELOPE AND ASSOCIATED CATEGORIES

ENVELOPE FUNCTIONS:
Transporters/membrane proteins (10 affected ORFS): Major facilitator
superfamily proteins, amino acid permeases, hypothetical membrane
protein, LemA membrane protein, intramembrane metalloprotease, AAA
ATPase, sodium-proton antiporter
Gain-of-function in penicillin-binding protein and amino acid permease
Cell wall/peptidoglycan synthesis: Penicillin-binding proteins, O-antigen
ligase domain-containing proteins (many mutations)
ENVELOPE MODIFYING FUNCTIONS:
Oxidation/reduction: Flavin reductase, alpha/beta hydrolase, pyruvate
carboxylase, MocA oxidoreductase, protophyringen oxidase, KGD
Glycosyl transferases/hydrolases: Glycosyl transferase, glycosyl
hydrolase, adenine glycosylase

These mutations primarily altered the structure and composition and function of the cellular envelope, which consists of the cell wall and membrane(s), including the cytoplasmic membrane. A complete list of the mutations identified is provided in Table 3. We did not see any mutations in genes that have been targeted for metabolic engineering and manipulated using recombinant DNA as previously reported (see Table 1). Mutations in multiple genes appear to be required to produce the mutant phenotype (such as increased growth in media supplemented with organic acid and/or overproduction of organic acid compared to the starting microbial cell line). This is in direct contrast to prior knowledge where single genes were manipulated to try to change PA yields.

In accordance with the present invention, other conventional microbiology, molecular biology, recombinant DNA, and biochemical techniques may be used. Such techniques are fully explained in the literature and within the skill of the art. The invention will be further described in the following examples, which do not limit the scope of the methods and compositions of matter recited in the claims.

### EXAMPLES

### Example 1

### Isolation of Strain 3-1

A P. acidipropionici (ATCC 25562) was grown to high cell density in 10 mL M24+2.0% glucose media. Serial dilutions of this culture (10⁰ to 10⁻³) were then plated on solid M24+2.0% glucose media, solidified with agar, supplemented with 1.0%, 2.0%, and 3.0% (w/v) PA, all neutralized to pH 7.0 using sodium hydroxide. Cells were also plated on solid M24+2.0% glucose media with no additional PA.

After a 5-day anaerobic incubation at 30° C., colony growth at the different PA concentrations was assessed. Three colonies grew on the 3% PA plate plated with undiluted cells; no colony grew on the 3% PA plates plated with diluted cells. The three colonies were isolated and re-streaked onto no-PA, 2.0% PA, and 3.0% PA plates (all neutralized to pH 7.0 using sodium hydroxide), along with freshly grown wild-type P. acidipropionici cells.

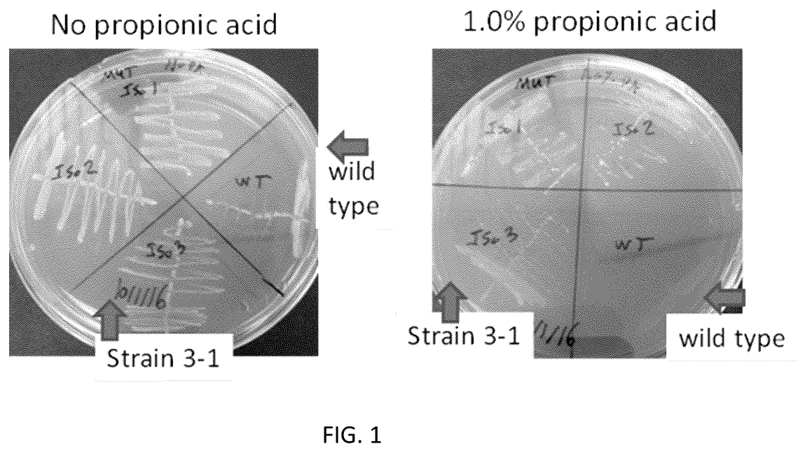

After a second 5-day anaerobic incubation at 30° C., colony growth at the different PA concentrations was again assessed. All three isolates, but not wild-type, were able to grow on the 1.0% PA plate (FIG. 1). Only isolate #1 was able to grow on the 2.0% PA and 3.0% PA plates. This isolate was named strain 3-1 (“3” denotes 3.0% PA, and “1” denotes isolate #1). Isolate #1 was inoculated into 5 mL liquid M24+2.0% glucose media and grown to high cell density, and frozen permanents of these cells were made.

After the phenotype of resistance to 3.0% PA on solid media was confirmed for strain 3-1, PA production in 10 mL batch cultures and 1 L bioreactor cultures of this strain was compared to its parental P. acidipropionici (ATCC 25562) cells by HPLC in a broad range of media and cultivation conditions.

Strain 3-1 was deposited under the name NFS-2018 on Jul. 10, 2019, in the American Type Culture Collection (10801 University Blvd. Manassas, Virginia 20110-2209) and assigned Accession Number ATCC PTA-125895).

### Example 2

### PA Production by Strain 3-1 and wild-type P. acidipropionici

Wild type P. acidipropionici (ATCC 255562) and strain 3-1 were cultivated from a frozen permanent at 30° C. under anaerobic condition in M24 medium supplemented with 2% glucose. The cells were sub-cultured every 48 hr into fresh M24 medium starting at 10 mL then at 50 mL to use as seed for the 1 L bioreactor vessels.

For preparation of wheat flour medium, 75 g of American cake flour was added to 1 L of ddH2O in a sterile 2 L flask while mixing. One mL of Enzenco alpha-amylase and 500 mL of 50 ppm of CaCl₂ was added to the mixture to hydrolyze the cake flour. The pH was adjusted to 6.0 by adding 5 mL of 5 M NaOH and the temperature was held at 90° C. for 1 hour. The mixture was allowed to cool then incubated at 37° C. overnight. After the overnight incubation, the temperature was raised to 60° C. and pH adjusted to 7.0 by adding 2 mL of 5 M NaOH. To release glucose, 1 mL of Enzenco glucoamylase, 0.05 g of protease, 0.4 g of MgSO4, and 10 g of Ohly KAT yeast extract were added to the mixture while stirring. The mixture was held at 60° C. for 2 hours. The mixture was allowed to cool then added to a glass-jacketed bioreactor vessel then sealed. Before autoclaving, the pH was calibrated.

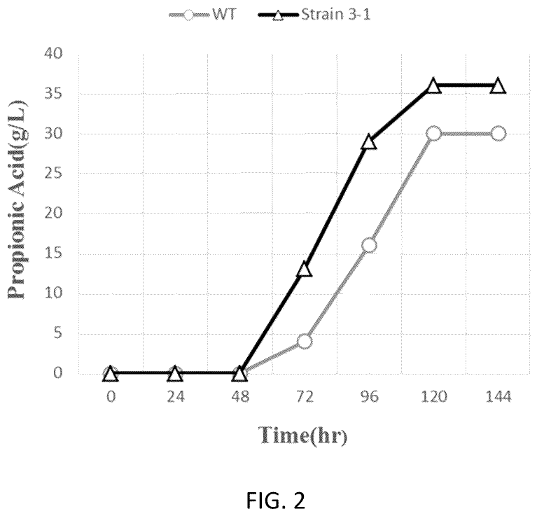

Fermentations were performed at 1 L volumes in the 3 L bioreactor vessels. The temperature was maintained at 30° C., the pH was maintained at 7.0 using 5 M NaOH, and cultures were agitated at 200 rpm. 3 mL of filtered sterile trace element solution was added to the bioreactor before inoculation. The glucose concentration was determined using a YSI 2900 analyzer. The 1 L of wheat flour medium was seeded with 5% inoculum. Samples were removed every 24 hours for PA analysis on the HPLC. The results are shown in FIG. 2.

Both strain 3-1 and the parental wild-type strain reached maximum PA concentration at about 120 hours. The maximum concentration of PA produced by strain 3-1 is about 36 g/L, compared to about 30 g/L by the parental wild-type strain.

Additional experiments were carried out under 5-6 different conditions, 3-4 times each, to compare PA production by strain 3-1 and wild-type P. acidipropionici. Results similar to those shown in FIG. 2 were obtained. There is a minimum of 15% increase in PA production by strain 3-1 compared to the wild-type after 60 hours of culturing.

### Example 3

### Genomic Analyses of Strain 3-1

Genome resequencing of strain 3-1 was used to identify the critical genes through their mutational changes that caused the genetic resistance to high concentrations of organic acids.

65 loss of function mutations in 29 genes were identified. The mutations generally affected cellular envelope functions, as shown in Table 2. These mutations primarily alter the structure and composition and function of the cellular envelope, which consists of the cell wall and membrane(s), including the cytoplasmic membrane. A complete list of the mutations identified in strain 3-1 is provided in Table 3.

TABLE 3
STRAIN 3-1 GENOME MUTATIONS

| | | SEQ | |
|---|---|---|---|
| | | ID | |
| Coordinates | ORF | NO. | Change |
| Non-synonymous | | | |
| 130744- | ASQ49_RS00690 | 1 | Arg → His |
| 130746 | class I SAM-dependent | | |
| | methyltransferase | | |
| 130744- | ASQ49_RS00695 | 2 | Thr → Pro |
| 130746 | MFS transporter | | |
| 130748 | ASQ49_RS00690 | 1 | Pro → Leu |
| | class I SAM-dependent | | |
| | methyltransferase | | |
| 130752 | ASQ49_RS00695 | 2 | Arg → Gly |
| | MFS transporter | | |
| 181601 | ASQ49_RS00915 | 3 | Insertion (no |
| (80% | LemA family protein | | frameshift) |
| confidence) | | | |
| 181607- | ASQ49_RS00915 | 3 | Gln → Leu |
| 181609 | LemA family protein | | |
| 240311 | ASQ49_01155 | 4 | Pro → His |
| | Flavin reductase | | |
| 240440 | ASQ49_01155 | 4 | Ala → Val |
| | Flavin reductase | | |
| 281222 | ASQ49_RS01330 | 5 | Trp → STOP |
| | Hypothetical protein | | |
| | (BLAST hit to MFS transporter) | | |
| 344598 | ASQ49_RS01635 | 6 | Thr → Pro |
| | MFS transporter | | |
| 525954 | ASQ49_RS02385 | 7 | Ala → Glu |
| | glycosyl transferase family 1 | | |
| 548143- | ASQ49_RS02475 | 8 | Ala → Gly |
| 548147 | Hypothetical protein | | Gly → Leu |
| | (Strong BLAST hits to O-antigen | | |
| | ligase and membrane protein) | | |
| 548153- | ASQ49_RS02475 | 8 | Ala → Val |
| 548156 | Hypothetical protein | | Gly → Leu |
| | (Strong BLAST hits to O-antigen | | |
| | ligase and membrane protein) | | |
| 548162 | ASQ49_RS02475 | 8 | Ala → Val |
| | Hypothetical protein | | |
| | (Strong BLAST hits to O-antigen | | |
| | ligase and membrane protein) | | |
| 558158- | ASQ49_RS02520 | 9 | His → Gly |
| 558160 | O-antigen ligase domain- | | |
| | containing protein | | |
| 558181 | ASQ49_RS02520 | 9 | Gln → His |
| | O-antigen ligase domain- | | |
| | containing protein | | |
| 558228 | ASQ49_RS02520 | 9 | Ala → Thr |
| | O-antigen ligase domain- | | |
| | containing protein | | |
| 558252 | ASQ49_RS02520 | 9 | Ser → Pro |
| | O-antigen ligase domain- | | |
| | containing protein | | |
| 558258 | ASQ49_RS02520 | 9 | Ser → Pro |
| | O-antigen ligase domain- | | |
| | containing protein | | |
| 558266 | ASQ49_RS02520 | 9 | Arg → Leu |
| | O-antigen ligase domain- | | |
| | containing protein | | |
| 558273 | ASQ49_RS02520 | 9 | Ser → Ala |
| | O-antigen ligase domain- | | |
| | containing protein | | |
| 558279 | ASQ49_RS02520 | 9 | Gln → Glu |
| | O-antigen ligase domain- | | |
| | containing protein | | |
| 558282 | ASQ49_RS02520 | 9 | Glu → Gln |
| | O-antigen ligase domain- | | |
| | containing protein | | |
| 558288- | ASQ49_RS02520 | 9 | Leu → Pro |
| 588290 | O-antigen ligase domain- | | |
| | containing protein | | |
| 558291- | ASQ49_RS02520 | 9 | Glu → Val |
| 558293 | O-antigen ligase domain- | | |
| | containing protein | | |
| 558306 | ASQ49_RS02520 | 9 | Pro → Ala |
| | O-antigen ligase domain- | | |
| | containing protein | | |
| 558308- | ASQ49_RS02520 | 9 | Thr → Ser |
| 558310 | O-antigen ligase domain- | | |
| | containing protein | | |
| 562835 | ASQ49_RS02535 | 10 | Val → Leu |
| | O-antigen ligase domain- | | |
| | containing protein | | |
| 562840 | ASQ49_RS02535 | 10 | Ala → Val |
| | O-antigen ligase domain- | | |
| | containing protein | | |
| 562843 | ASQ49_RS02535 | 10 | Gly → Ala |
| | O-antigen ligase domain- | | |
| | containing protein | | |
| 566353 | ASQ49_RS02550 | 11 | Lys → Gln |
| (*50% | penicillin-binding protein | | |
| frequency) | | | |
| 566356 | ASQ49_RS02550 | 11 | Ala → Ser |
| (*50% | penicillin-binding protein | | |
| frequency) | | | |
| 618017- | ASQ49_RS02820 | 12 | Glu → Ala |
| 618019 | Phosphotransferase | | |
| 738302 | ASQ49_RS03340 | 13 | Thr → Ala |
| | Alpha/beta hydrolase | | |
| 742073 | ASQ49_RS03360 | 14 | Ile → Leu |
| | Hypothetical protein | | |
| | (BLAST hits to intramembrane | | |
| | metalloprotease) | | |
| 1176596 | ASQ49_RS05220 | 15 | Ala → Val |
| | gfo/Idh/MocA family | | |
| | oxidoreductase | | |
| 1279986 | ASQ49_RS05630 | 16 | Ile → Val |
| | Alpha/beta hydrolase | | |
| 1331356 | ASQ49_RS05840 | 17 | Gly → Ser |
| | Amino acid permease | | |
| 1331366 | ASQ49_RS05840 | 17 | Arg → His |
| | Amino acid permease | | |
| 1521847 | ASQ49_RS06625 | 18 | Thr → Ala |
| | Hypothetical protein | | |
| | (BLAST hits to | | |
| | protoporphyrinogen oxidase) | | |
| 1816621 | ASQ49_RS07985 | 19 | Ser → Ala |
| | Adenine glycosylase | | |
| 1816687 | ASQ49_RS07985 | 19 | In-frame insertion |
| | Adenine glycosylase | | (1 amino acid) |
| 1817191 | ASQ49_RS07985 | 19 | Gly → Glu |
| | Adenine glycosylase | | |
| 1817202 | ASQ49_RS07985 | 19 | Glu → Ala |
| | Adenine glycosylase | | |
| 1854503 | SQ49_RS08150 | 20 | Lys → Arg |
| | Hypothetical protein | | |
| | (BLAST hit to sodium-proton | | |
| | antiporter) | | |
| 1854520 | SQ49_RS08150 | 20 | Ile → Met |
| | Hypothetical protein | | |
| | (BLAST hit to sodium-proton | | |
| | antiporter) | | |
| 2679601 | ASQ49_12020 | 21 | His → Asp |
| | multifunctional oxoglutarate | | |
| | decarboxylase/oxoglutarate | | |
| | dehydrogenase thiamine | | |
| | pyrophosphate-binding | | |
| | subunit/dihydrolipoyllysine- | | |
| | residue succinyltransferase | | |
| | subunit (kgd) | | |
| 2927020 | ASQ49_RS13125 | 22 | In-frame insertion |
| | Amino acid permease | | (4 amino acids) |
| 2927030 | ASQ49_RS13125 | 22 | Gly-Ser → Ala-Ala |
| | Amino acid permease | | |
| 2928883 | ASQ49_RS13130 | 23 | Asn → Tyr |
| | Hypothetical protein | | |
| | (glycosyl gydrolase family) | | |
| 3517645 | ASQ49_RS15965 | 24 | Thr → Arg |
| (*50% | M18 family aminopeptidase | | |
| frequency) | | | |
| 3517646 | ASQ49_RS15965 | 24 | Thr → Ser |
| (*50% | M18 family aminopeptidase | | |
| frequency) | | | |
| 3517648 | ASQ49_RS15965 | 24 | Ser → Thr |
| (*50% | M18 family aminopeptidase | | |
| frequency) | | | |
| 3517649 | ASQ49_RS15965 | 24 | Ser → Gly |
| (*50% | M18 family aminopeptidase | | |
| frequency) | | | |
| 3517652 | ASQ49_RS15965 | 24 | Ser → Tyr |
| | M18 family aminopeptidase | | |
| 3517655 | ASQ49_RS15965 | 24 | Ser → Asn |
| | M18 family aminopeptidase | | |
| FRAMESHIFTS | | | |
| 558244 | ASQ49_RS02520 | 9 | |
| | O-antigen ligase domain- | | |
| | containing protein | | |
| 558246 | ASQ49_RS02520 | 9 | |
| | O-antigen ligase domain- | | |
| | containing protein | | |
| 2867178 | ASQ49_RS12835 | 25 | |
| | AAA ATPase | | |
| FRAMESHIFT | | | |
| REPAIRS | | | |
| 448285 | ASQ49_RS02075 | 26 | |
| | DUF1116 domain-containing | | |
| | protein | | |
| 561527 | ASQ49_RS02530 | 27 | |
| | Glycosyl transferase | | |
| 900222 | ASQ49_RS03980 | 28 | |
| | acetyl-CoA carboxylase biotin | | |
| | carboxyl carrier protein subunit | | |
| 919056 | ASQ49_RS04070 | 29 | |
| | Penicillin-binding protein | | |
| 1330401 | ASQ49_RS05840 | 17 | |
| | Amino acid permease | | |
| 1330407 | ASQ49_RS05840 | 17 | |
| | Amino acid permease | | |

Mutations in these genes (or their homologues in other species described herein) likely confer genetic resistance to high concentrations of organic acids by altering the membrane transport protein systems and/or previously unknown intracellular targets involved in regulation and/or metabolism.

Multiple mutations in the same gene imply that the gene is very important for the trait and required multiple changes to contribute to the trait. Noticeably, several genes had three or more mutations, which may indicate their critical roles in limiting organic acid formation. They include genes encoding: O-antigen ligase domain-containing protein (15 mutations in ASQ49_RS02520; 3 mutations in ASQ49_RS02535; and 3 mutations in ASQ49_RS02475 (hypothetical protein with strong BLAST hits to O-antigen ligase and membrane protein)); M18 family aminopeptidase (6 mutations in ASQ49_RS15965); amino acid permease (4 mutations in ASQ49_RS05840); and adenine glycosylase (4 mutations in ASQ49_RS07985).

### REFERENCES

1. Woskow S.A., B.A. Glatz. 1991. Propionic acid production by a propionic acid-tolerant strain of Propionibacterium acidipropionici in batch and semicontinuous fermentation. Applied and Environmental Microbiology 57:2821-2828.
2. Zhu Y., J. Li, M. Tan, L. Liu, L. Jiang, J. Sun, P. Lee, G. Du, J. Chen. 2010. Optimization and scale-up of propionic acid production by propionic acid-tolerant Propionibacterium acidipropionici with glycerol as the carbon source. Bioresource Technology 101:8902-8906.
3. Wang Z., S.-T. Yang. 2013. Propionic acid production in glycerol/glucose co-fermentation by Propionibacterium freudenreichii subsp. shermanii. Bioresource Technology 137:116-123.
4. Zhuge X., L. Liu, H.-d. Shin, J. Li, G. Du, J. Chen. 2014. Improved propionic acid production from glycerol with metabolically engineered Propionibacterium jensenii by integrating fed-batch culture with a pH-shift control strategy. Bioresource Technology 152:519-525.
5. Zhuge X., J. Li, H.-d. Shin, L. Liu, G. Du, J. Chen. 2015. Improved propionic acid production with metabolically engineered Propionibacterium jensenii by an oxidoreduction potential-shift control strategy. Bioresource Technology 175:606-612.
6. Coral J. 2008. Propionic acid production by Propionibacterium sp. using low-cost carbon sources in submerged fermentation. Dissertation. Federal University of Parana.
7. Zhang A., J. Sun, Z. Wang, S.-T. Yang, H. Zhou. 2015. Effects of carbon dioxide on cell growth and propionic acid production from glycerol and glucose by Propionibacterium acidipropionici. Bioresource Technology 175:374-381.
8. Wang Z., M. Lin, L. Wang, E. M. Ammar, S.-T. Yang. 2015. Metabolic engineering of Propionibacterium freudenreichii subsp. shermanii for enhanced propionic acid fermentation: Effects of overexpressing three biotin-dependent carboxylases. Process Biochemistry 50:194-204.
9. Suwannakham S., Y. Huang, S.-T. Yang. 2006. Construction and characterization of ack knock-out mutants of Propionibacterium acidipropionici for enhanced propionic acid fermentation. Biotechnology and Bioengineering 94:383-95.
10. Suwannakham S., S.-T. Yang. 2005. Enhanced propionic acid fermentation by Propionibacterium acidipropionici mutant obtained by adaptation in a fibrous-bed bioreactor. Biotechnology and Bioengineering 91:325-337.
11. Suwannakham S. 2005. Metabolic engineering for enhanced propionic acid fermentation by Propionibacterium acidipropionici. Dissertation. Ohio State University.
12. Tufvesson P., A. Ekman, R. R. R. Sardari, K. Engdahl, L. Tufvesson. 2013. Economic and environmental assessment of propionic acid production by fermentation using different renewable raw materials. Bioresource Technology 149:556-564.
13. Thierry A., S.-M. Deutsch, H. Falentin, M. Dalmasso, F. J. Cousin, G. January 2011. New insights into physiology and metabolism of Propionibacterium freudenreichii. International Journal of Food Microbiology 149:19-27.
14. Scholz C.F.P., M. Kilian. 2016. The natural history of cutaneous propionibacteria, and reclassification of selected species within the genus Propionibacterium to the proposed novel genera Acidipropionibacterium gen. nov., Cutibacterium gen. nov. and Pseudopropionibacterium gen. nov. International Journal of Systematic and Evolutionary Microbiology 66:4422-4432.
15. Rehberger J.L., B.A. Glatz. 1998. Response of cultures of Propionibacterium to acid and low pH: tolerance and inhibition. Journal of Food Production 61:211-216.
16. Ahmadi N., K. Khosravi-Darani, A.M. Mortazavian. 2017. An overview of biotechnological production of propionic acid: From upstream to downstream processes. Electronic Journal of Biotechnology 28:67-75.
17. Guan N., L. Liu, X. Zhug, Q. Xu, J. Li, G. Du, J. Chen. 2012. Genome-shuffling improves acid tolerance of Propionibacterium acidipropionici and propionic acid production. Advances in Chemistry Research 15:143-152.
18. Guan N., H. Shin, R. R. Chen, J. Li, L. Liu, G. Du, J. Chen. 2014. Understanding of how Propionibacterium acidipropionici respond to propionic acid stress at the level of proteomics. Scientific Reports 4:6951.
19. Guan N., H.D. Shin, G. Du, J. Chen, L. Liu. 2016. Metabolic engineering of acid resistance elements to improve acid resistance and propionic acid production of Propionibacterium jensenii. Biotechnology and Bioengineering 113:1294-304.
20. Jiang L., H. Cui, L. Zhu, Y. Hu, X. Xu, S. Li, H. Huang. 2015. Enhanced propionic acid production from whey lactose with immobilized Propionibacterium acidipropionici and the role of trehalose synthesis in acid tolerance. Green Chemistry 15:250-259.
21. EP 0141642
22. WO 85/04901
23. US 2011/0151529 A1
24. WO 2012/064883 A2
25. WO 2017/055932 A2

1. A method of producing an organic acid selected from propionic acid, lactic acid, acetic acid, butyric acid, or combinations thereof, the method comprising:
providing a P. acidipropionici cell comprising one or more loss of function mutations in genes encoding DUF1116 domain-containing protein comprising the amino acid sequence of SEQ ID NO:26 and M18 family aminopeptidase comprising the amino acid sequence SEQ ID NO:24; and
culturing the cell in a culture medium.

2. The method of claim 1, wherein the culture medium is maintained at a pH of from about 5.5-7.5.

3. The method of claim 2, wherein the culture medium is maintained at a pH of from about 6.0-7.0.

4. The method of claim 1, wherein said culturing is carried out at about 30° C. for about 60 to about 120 hours.

5. The method of claim 1, wherein the P. acidipropionici cell further comprises one or more additional mutations in a gene encoding O-antigen ligase domain-containing protein comprising the amino acid sequence of SEQ ID NO:9; adenine glycosylase comprising the amino acid sequence of SEQ ID NO:19; class I SAM-dependent methyltransferase comprising the amino acid sequence of SEQ ID NO: 1, MFS transporter comprising the amino acid sequence of SEQ ID NO: 2, LemA family protein comprising the amino acid sequence of SEQ ID NO: 3, Flavin reductase comprising the amino acid sequence of SEQ ID NO: 4, comprising the amino acid sequence of SEQ ID NO: 5, MFS transporter comprising the amino acid sequence of SEQ ID NO: 6, glycosyl transferase family 1 comprising the amino acid sequence of SEQ ID NO: 7, protein comprising the amino acid sequence of SEQ ID NO: 8, O-antigen ligase domain-containing protein comprising the amino acid sequence of SEQ ID NO: 10, penicillin-binding protein comprising the amino acid sequence of SEQ ID NO: 11, phosphotransferase comprising the amino acid sequence of SEQ ID NO: 12. alpha/beta hydrolase comprising the amino acid sequence of SEQ ID NO:13, protein comprising the amino acid sequence of SEQ ID NO:14, gfo/Idh/MocA family oxidoreductase comprising the amino acid sequence of SEQ ID NO: 15, alpha/beta hydrolase comprising the amino acid sequence of SEQ ID NO: 16, protein comprising the amino acid sequence of SEQ ID NO: 18, protein comprising the amino acid sequence of SEQ ID NO:20, multifunctional oxoglutarate decarboxylase/oxoglutarate dehydrogenase thiamine pyrophosphate-binding subunit/dihydrolipoyllysine-residue succinyltransferase subunit comprising the amino acid sequence of SEQ ID NO:21, amino acid permease comprising the amino acid sequence of SEQ ID NO:22, protein comprising the amino acid sequence of SEQ ID NO:23, ATPase comprising the amino acid sequence of SEQ ID NO:25, glycosyl transferase comprising the amino acid sequence of SEQ ID NO:27, acetyl-Co A carboxylase biotin carboxyl carrier protein subunit comprising the amino acid sequence of SEQ ID NO:28, and penicillin-binding protein comprising the amino acid sequence of SEQ ID NO:29.

6. The method of claim 1, wherein the P. acidipropionici cell comprises a loss of function mutation in each of the genes encoding the proteins DUF1116 domain-containing protein comprising the amino acid sequence of SEQ ID NO: 26, M18 family aminopeptidase comprising the amino acid sequence SEQ ID NO:24, O-antigen ligase domain-containing protein comprising the amino acid sequence of SEQ ID NO:9. adenine glycosylase comprising the amino acid sequence of SEQ ID NO: 19, class I SAM-dependent methyltransferase comprising the amino acid sequence of SEQ ID NO: 1, MFS transporter comprising the amino acid sequence of SEQ ID NO: 2, LemA family protein comprising the amino acid sequence of SEQ ID NO: 3, Flavin reductase comprising the amino acid sequence of SEQ ID NO: 4, comprising the amino acid sequence of SEQ ID NO: 5, MFS transporter comprising the amino acid sequence of SEQ ID NO: 6, glycosyl transferase family 1 comprising the amino acid sequence of SEQ ID NO: 7, protein comprising the amino acid sequence of SEQ ID NO: 8, O-antigen ligase domain-containing protein comprising the amino acid sequence of SEQ ID NO: 10, penicillin-binding protein comprising the amino acid sequence of SEQ ID NO: 11, phosphotransferase comprising the amino acid sequence of SEQ ID NO: 12. alpha/beta hydrolase comprising the amino acid sequence of SEQ ID NO: 13, protein comprising the amino acid sequence of SEQ ID NO: 14, gfo/Idh/MocA family oxidoreductase comprising the amino acid sequence of SEQ ID NO: 15, alpha/beta hydrolase comprising the amino acid sequence of SEQ ID NO: 16, protein comprising the amino acid sequence of SEQ ID NO: 18, protein comprising the amino acid sequence of SEQ ID NO:20, multifunctional oxoglutarate decarboxylase/oxoglutarate dehydrogenase thiamine pyrophosphate-binding subunit/dihydrolipoyllysine-residue succinyltransferase subunit comprising the amino acid sequence of SEQ ID NO:21, amino acid permease comprising the amino acid sequence of SEQ ID NO:22, protein comprising the amino acid sequence of SEQ ID NO:23, ATPase comprising the amino acid sequence of SEQ ID NO:25, glycosyl transferase comprising the amino acid sequence of SEQ ID NO:27, acetyl-Co A carboxylase biotin carboxyl carrier protein subunit comprising the amino acid sequence of SEQ ID NO:28, and penicillin-binding protein comprising the amino acid sequence of SEQ ID NO:29.

7. The method of claim 1, wherein the P. acidipropioni cell is the P. acidipropioni cell deposited in the American Type Culture Collection under Accession Number ATCC PTA-125895.

8. The method of claim 1, wherein the organic acid is propionic acid.

9. The method of claim 1, wherein the amount of organic acid produced by the method after 60 hours of culturing is at least 15% higher as compared to the amount of organic acid produced under the same conditions using wild-type P. acidipropionici.

10-16. (canceled)
